(19) United States
(12) Patent Application Publication
(10) Pub. No.: US 2025/0228876 A1
(43) Pub. Date: Jul. 17, 2025

(54) COMPOSITIONS FOR DELIVERY OF CANNABINOIDS

(71) Applicant: PNEUMA RESPIRATORY, INC., Boone, NC (US)

(72) Inventor: Matthew Culpepper, Boone, NC (US)

(73) Assignee: PNEUMA RESPIRATORY, INC., Boone, NC (US)

(21) Appl. No.: 18/700,520

(22) PCT Filed: Oct. 13, 2022
(86) PCT No.: PCT/US2022/046582

§ 371 Date: Apr. 11, 2024

Related U.S. Application Data
(60) Provisional application No. 63/255,326, filed on Oct. 13, 2021.

(51) Int. Cl. A61K 31/00; A61K 9/00; A61K 47/10

(52) U.S. Cl. CPC A61K 31/658; A61K 9/0073; A61K 47/10

(57) ABSTRACT

Embodiments of the present disclosure provide compositions and methods for administering cannabinoids to the respiratory system of a subject. Accordingly, embodiments herein include compositions comprising one or more cannabinoids and pharmaceutical compositions comprising one or more cannabinoids. Embodiments herein also include methods of making compositions herein, methods of delivering compositions herein, and methods of treating a subject by administering one or more compositions herein.

### CROSS-REFERENCE TO RELATED APPLICATIONS

The present application claims the benefit of priority of U.S. Provisional Patent Application No. 63/255,326, filed Oct. 13, 2021, the disclosures of which are incorporated herein by reference in their entirety.

### FIELD

Embodiments of the instant disclosure generally relate to compositions and methods for administering cannabinoids and cannabinoid-containing compositions to the respiratory system.

### BACKGROUND

A growing body of evidence supports that cannabinoids have important medical uses and applications. As acceptance of the medicinal properties of cannabinoids grows, there is a need to identify routes of administration that can improve cannabinoid bioavailability and establish therapeutic efficacy, dose ranges, safety and also improve the patient compliance. Accordingly, what is needed for optimal use of medicinal cannabinoids is a feasible, nonsmoked, rapid-onset delivery system for local administration to the respiratory system while avoiding the respiratory disadvantages of smoking. However, to date, targeted respiratory delivery of cannabinoids has not been thoroughly investigated.

The use of droplet generating devices for the delivery of substances to the respiratory system is an area of large interest. A major challenge is providing a device that delivers an accurate, consistent, and verifiable amount of substance, with a droplet size that is suitable for successful delivery of the substance to the targeted area of the respiratory system. Currently most inhaler type systems, such as metered dose inhalers (MDI), pressurized metered dose inhalers (p-MDI), or pneumatic and ultrasonic-driven nebulizer devices, generally produce droplets are not suited for delivery of many substances. Such devices generate droplets with high velocities and a wide range of droplet sizes, including large droplets that have high momentum and kinetic energy. Droplet plumes with large size distributions and high momentum do not reach a targeted area in the respiratory system, but rather deposit throughout the pulmonary passageways, mouth and throat. Such non-targeted deposition may be undesirable for many reasons, including improper dosing and unwanted side effects.

Accordingly, there is a need for an improved method to deliver cannabinoids to the respiratory system, wherein the droplets are delivered in a targeted manner.

### SUMMARY

Embodiments of the present disclosure relate to methods and compositions for administering at least one cannabinoid to the respiratory system of a subject. In certain embodiments, the present disclosure provides compositions that may comprise one or more cannabinoids, propylene glycol, ethanol, water, or any combination thereof. In some embodiments, compositions herein may comprise a cannabinoid in a concentration of about 1 mg/mL to about 20 mg/mL; propylene glycol in a concentration of about 0.1 g/mL to about 0.3 g/mL; ethanol in a concentration of about 0.2 g/mL to about 0.4 g/mL; and water, wherein the composition may have a surface tension of about 20 mN/m to about 35 mN/m and the composition may be a solution.

In some embodiments, compositions herein may have a surface tension of about 25 mN/m to about 34 mN/m. In some embodiments, compositions herein may have a surface tension of about 31 mN/m to about 33 mN/m.

In some embodiments, compositions herein may further comprise glycerol. In some embodiments, compositions herein may further comprise glycerol in a concentration of about 35 mg/mL to about 50 mg/mL. In some embodiments, compositions herein may further comprise glycerol wherein the glycerol may be present in an amount of about 40 mg/mL to about 50 mg/mL.

In some embodiments, compositions herein may further comprise glycerin. In some embodiments, compositions herein may further comprise glycerin wherein the glycerin may have a glycerol concentration of at least about 95%. In some embodiments, compositions herein may further comprise glycerin wherein the glycerin may have a glycerol concentration of at least about 99%. In some embodiments, compositions herein may comprise one or more cannabinoids selected from tetrahydrocannabinol (THC), cannabidiol (CBD), cannibichromene (CBC), cannabigerol (CBG), cannabinol (CBN), cannabichromevarin (CBCV), cannabichromevarin (CBCV), cannabidiphorol (CBDP), cannabielsoin (CBE), cannabigerol (CBG), Cannabicyclol (CBL), Cannabinol (CBN), cannabicitran (CBT), cannabigerovarin (CBGV), cannabigerol monomethyl ether (CBGM), Cannabivarin (CBV), delta-8-tetrahydrocannabinol (delta-8-THC, Δ8-THC), (−)-trans-Δ9-tetrahydrocannabiphorol (Δ9-THCP, (C7)-Δ9-THC, and THC-Heptyl), Δ9-tetrahydrocannabiorcol (Δ9-THCC, (C1)-Δ9-THC), tetrahydrocannabivarin (THCV, THV), dimethylheptylpyran, parahexyl, or any combination thereof.

In some embodiments, compositions herein may comprise a cannabinoid wherein the cannabinoid may be cannabidiol (CBD).

In some embodiments, compositions herein may comprise a cannabinoid wherein the cannabinoid may be tetrahydrocannabinol (THC).

In some embodiments, compositions herein may comprise a cannabinoid wherein the cannabinoid may be at least 90% pure. In some embodiments, compositions herein may comprise a cannabinoid wherein the cannabinoid may be at least 95% pure. In some embodiments, compositions herein may comprise a cannabinoid wherein the cannabinoid may be at least 99% pure.

In some embodiments, compositions herein may comprise propylene glycol in an amount of about 0.15 g/mL to about 0.25 g/mL. In some embodiments, compositions herein may comprise propylene glycol in an amount of about 0.17 g/mL to about 0.24 g/mL. In some embodiments, compositions herein may comprise a propylene glycol that may be Food Chemicals Codes (FCC) and/or Food Grade (FG).

In some embodiments, compositions herein may comprise ethanol in an amount of about 0.24 g/mL to about 0.33 g/mL. In some embodiments, compositions herein may comprise an ethanol that may be about 180 proof to about 200 proof. In some embodiments, compositions herein may comprise an ethanol that may be about 200 proof.

In some embodiments, compositions herein may comprise water in an amount of about 0.3 g/mL to about 0.6 g/mL. In some embodiments, compositions herein may comprise water wherein the water may be deionized water, distilled water, or a combination thereof.

In some embodiments, compositions herein may be a composition for inhalation by a subject. In some embodiments, compositions herein may be delivered to the respiratory system of a subject.

In some embodiments, compositions herein may be a pharmaceutical composition for use in treating one or more of epilepsy, multiple sclerosis (MS), post-traumatic stress disorder (PTSD), schizophrenia, fibromyalgia, Alzheimer's disease, dementia, Parkinson's disease, Crohn's disease, glaucoma, cancer, HIV/AIDS, substance use disorder, or any combination thereof.

In certain embodiments, the present disclosure provides methods of making any one of the compositions disclosed herein. In some embodiments, methods of making a composition herein may include one or more of the following: (a) adding a propylene glycol to a cannabinoid and stirring until combined to form a propylene glycol/cannabinoid mixture; (b) adding an ethanol to the propylene glycol/cannabinoid mixture to form an ethanol/propylene glycol/cannabinoid mixture; and (c) adding water in increments to the ethanol/propylene glycol/cannabinoid mixture while stirring until the full amount of water is added, thereby making the composition. In some embodiments, methods of making a composition herein may include dissolving the cannabinoid before adding the water in (c). In some embodiments, methods of making a composition herein may be performed at temperatures equal to or higher than about 20° C.

In certain embodiments, the present disclosure provides methods of delivering any one of the compositions disclosed herein to the respiratory system of a user. In some embodiments, methods herein may deliver a composition disclosed herein as an ejected stream of droplets in a respirable range to the respiratory system of a user. In some embodiments, methods herein may deliver a composition disclosed herein as an ejected stream of droplets in a respirable range to the respiratory system of a user, the method comprising: (a) generating an ejected stream of droplets from the fluid composition via a droplet delivery device comprising an ejector mechanism having a piezoelectric actuator, and an aperture plate, the aperture plate having a plurality of openings formed through its thickness and the piezoelectric actuator being operable to directly or indirectly oscillate the aperture plate at a frequency to thereby generate the ejected stream of droplets, wherein at least about 50% of the ejected stream of droplets may have an average ejected droplet diameter of less than about 6 μm; and (b) delivering the ejected stream of droplets to the respiratory system of the user such that at least about 50% of the mass of the ejected stream of droplets is delivered in a respirable range to the respiratory system of a user during use.

In some embodiments, methods herein may deliver a composition disclosed herein as an ejected stream of droplets (droplet stream) in a respirable range to the respiratory system of a user, the method comprising: (a) generating a droplet stream from the fluid composition via an droplet delivery device wherein the droplet stream is produced by the droplet delivery device by delivering a fluid volume between a membrane and mesh, electronically activating an ultrasonic transducer coupled to the membrane via vibrating member and pushing the fluid volume through openings in the mesh to produce the droplet stream; and (b) delivering the droplet stream to the respiratory system of the user such that at least about 50% of the mass of the droplet stream is delivered in a respirable range to the respiratory system of a user during use.

In some embodiments, methods herein may deliver a composition comprising cannabidiol (CBD) to a user. In some embodiments, methods herein may deliver a composition comprising CBD to a user to treat or ameliorate one or more diseases, conditions or disorders selected from the group consisting of epilepsy, multiple sclerosis (MS), post-traumatic stress disorder (PTSD), schizophrenia, fibromyalgia, Alzheimer's disease, dementia, Parkinson's disease, Crohn's disease, glaucoma, cancer, HIV/AIDS, and substance use disorder. In some embodiments, methods herein may deliver a composition comprising CBD to a user to treat or ameliorate one or more diseases, conditions or disorders selected from the group consisting of nausea, vomiting, loss of appetite, pain, insomnia, migraine, muscle spasm, seizure, and anxiety.

In some embodiments, methods herein may deliver a composition comprising tetrahydrocannabinol (THC) to a user. In some embodiments, methods herein may deliver a composition comprising THC to a user to treat or ameliorate one or more diseases, conditions or disorders selected from the group consisting of epilepsy, multiple sclerosis (MS), post-traumatic stress disorder (PTSD), schizophrenia, fibromyalgia, Alzheimer's disease, dementia, Parkinson's disease, Crohn's disease, glaucoma, cancer, HIV/AIDS, and substance use disorder. In some embodiments, methods herein may deliver a composition comprising THC to a user to treat or ameliorate one or more diseases, conditions or disorders selected from the group consisting of nausea, vomiting, loss of appetite, pain, insomnia, migraine, muscle spasm, seizure, and anxiety.

In some embodiments, methods herein may deliver a composition via a droplet

delivery device comprising an ejector mechanism, wherein the aperture plate (or mesh) of the ejector mechanism may have at least a fluid entrance side of one or more of said plurality of openings configured so as to provide a surface contact angle of less than about 90 degrees. In accordance with some embodiments herein, the aperture plate (or mesh) of the ejector mechanism may be configured such that at least the fluid entrance side of one or more of said plurality of openings is configured to provide a surface contact angle of between 2 and 80 degrees. In accordance with some embodiments herein, at least a portion of the interior of the plurality of openings is configured so as to provide a surface contact angle of less than 90 degrees.

In some embodiments, a surface contact angle of less than 90 degrees at the fluid entrance side of one or more of said plurality of openings may be obtained by surface coating with a hydrophilic material, surface structural modification, or a combination thereof. In accordance with some embodiments herein, the hydrophilic material may be selected from siloxane based coatings, isocyante based coatings, ethylene oxide based coatings, polyisocyanate based coatings, hydrocyclosiloxane based coatings, hydroxyalkylmethacrylate based coatings, hydroxyalkylacrylate based coatings, glycidylmethacrylate based coatings, propylene oxide based coatings, N-vinyl-2-pyrrolidone based coatings, latex based coatings, polyvinylchloride based coatings, and/or polyurethane based coatings.

In some embodiments, methods herein of delivering a composition disclosed herein as an ejected stream of droplets may have at least about 50% of the ejected stream of droplets having an average ejected droplet diameter of less than about 4.0 μm. In some embodiments, methods herein of delivering a composition disclosed herein as an ejected stream of droplets may have at least about 50% of the ejected stream of droplets having an average ejected droplet diameter between about 0.7 μm and about 3.2 μm.

In some embodiments, methods herein of delivering a composition disclosed herein as an ejected stream of droplets may deliver the ejected stream of droplets over a period of time less than about 2 seconds.

### Definitions

Terms, unless defined herein, have meanings as commonly understood by a person of ordinary skill in the art relevant to certain embodiments disclosed herein or as applicable.

Unless otherwise indicated, all numbers expressing quantities of agents and/or compounds, properties such as molecular weights, reaction conditions, and as disclosed herein are contemplated as being modified in all instances by the term “about.” Accordingly, unless indicated to the contrary, the numerical parameters in the specification and claims are approximations that can vary from about 10% to about 15% plus and/or minus depending upon the desired properties sought as disclosed herein. Numerical values as represented herein inherently contain standard deviations that necessarily result from the errors found in the numerical value's testing measurements.

As used herein, “individual”, “subject”, “host”, and “patient” are used interchangeably herein and refer to any mammalian subject for whom diagnosis, treatment, prophylaxis or therapy is desired, particularly humans.

As used herein, “treat,” “treating” or “treatment” can refer to treating, reversing, ameliorating, or inhibiting onset or inhibiting progression of a health condition or disease or a symptom of the health condition or disease.

Unless otherwise defined, all technical terms used herein have the same meaning as commonly understood by one of ordinary skill in the art to which this disclosure belongs.

### DETAILED DESCRIPTION

In the following sections, certain exemplary compositions and methods are described in order to detail certain embodiments of the invention. It will be obvious to one skilled in the art that practicing the certain embodiments does not require the employment of all or even some of the specific details outlined herein, but rather that concentrations, times and other specific details can be modified through routine experimentation. In some cases, well known methods, or components have not been included in the description.

In certain aspects, the present disclosure generally relates to methods for delivering a fluid composition comprising at least one cannabinoid as an ejected stream of droplets in a respirable range to the respiratory system of a user. In certain aspects, the fluid composition comprising at least one cannabinoid may be delivered at a high dose concentration and efficacy, as compared to alternative dosing routes and standard inhalation technologies. In some aspects, the fluid composition comprising at least one cannabinoid may be delivered to the user with lower levels of contaminants, undesired compounds, etc. as compared to alternative dosing routes and standard inhalation technologies.

### I. Compositions

Certain embodiments herein provided for compositions comprising at least one cannabinoid. In some embodiments, compositions herein may comprise at least one cannabinoid and at least one pharmaceutically acceptable carrier or diluent. In some embodiments, the compositions herein may be solutions. In some embodiments, compositions herein may be solutions for delivery of at least one cannabinoid to the respiratory system of a subject.

### (a) Cannabinoids

In some embodiments, compositions disclosed herein may encompass at least one cannabinoid in a concentration of about 1 mg/mL to about 20 mg/mL (e.g., about 1, 2, 3, 4, 5, 6, 7, 8, 9, 10, 11, 12, 13, 14, 15, 16, 17, 18, 19, 20 mg/mL). In some embodiments, compositions disclosed herein may encompass a total amount of two or more cannabinoids at a concentration of about 1 mg/mL to about 20 mg/mL (e.g., about 1, 2, 3, 4, 5, 6, 7, 8, 9, 10, 11, 12, 13, 14, 15, 16, 17, 18, 19, 20 mg/mL).

In some embodiments, compositions disclosed herein may encompass about 1% to about 80%, about 5% to about 50%, or about 10% to about 40% cannabinoid(s) by weight of the composition. In some embodiments, compositions disclosed herein may encompass about 1%, about 5%, about 10%, about 20%, about 30%, about 40%, about 50%, about 60%, about 70%, or about 80% cannabinoid(s) by weight of the composition.

In some embodiments, compositions disclosed herein may comprise at least one cannabinoid having at least about 85% purity. In some embodiments, compositions disclosed herein may comprise at least one cannabinoid having about 85% to 99% (e.g., about 85%, 90%, 95%, 96%, 97%, 98%, 99%) purity. In some embodiments, compositions disclosed herein may comprise at least one pure cannabinoid. Purity of cannabinoids herein may be determined using methods known in the art, including but not limited to high-performance liquid chromatography (HPLC).

In some embodiments, compositions disclosed herein may encompass at least one phytocannabinoid. As used herein, the term “phytocannabinoid” refers to a plant-based cannabinoid found in plants belonging to the genus Cannabis.

In some embodiments, compositions disclosed herein may encompass at least one cannabinoid in the cannabigerol class. Non-limiting examples of compounds comprising the cannabigerol class suitable for use herein can include cannabigerolic acid (CBGA), cannabigerolic acid monoethylether (CBGAM), cannabigerol monoethylether (CBGM), cannabigerol (CBG), cannabigerovarinic acid (CBGVA), and cannabigerovarin (CBGV).

In some embodiments, compositions disclosed herein may encompass at least one cannabinoid in the cannabichromene class. Non-limiting examples of compounds comprising the cannabichromene class suitable for use herein can include cannabichromene (CBC), cannabichromenic acid (CBCA), cannabichromevarinic acid (CBCVA), and cannabichromevarin (CBCV).

In some embodiments, compositions disclosed herein may encompass at least one cannabinoid in the cannabidiol class. Non-limiting examples of compounds comprising the cannabidiol class suitable for use herein can include cannabidiol (CBD), cannabidiolic acid (CBDA), cannabidiol monomethylether (CBDM), cannabidiol-C₄ (CBD-C₄), cannabidivarinic acid (CBDVA), cannabidivarin (CBDV), and cannabidiorcol (CBD-C₁).

In some embodiments, compositions disclosed herein may encompass at least one cannabinoid in the tetrahydrocannabinol class. In accordance with some embodiments herein, compounds comprising the tetrahydrocannabinol class may be in a delta-9-tetrahydrocannabinol class or a delta-8-tetrahydrocannabinol class. Non-limiting examples of compounds comprising the delta-9-tetrahydrocannabinol class suitable for use herein can include delta-9-tetrahydrocannabinolic acid A (THCA-A), delta-9-tetrahydrocannabinolic acid B (THCA-B), delta-9-tetrahydrocannabinol (THC), delta-9-tetrahydrocannabinolic acid-C₄ (THCA-C₄), delta-9-tetrahydrocannabinol-C₄ (THC-C₄), delta-9-tetrahydrocannabivarinic acid (THCVA), delta-9-tetrahydrocannabivarin (THCV), delta-9-tetrahydrocannabiorcolic acid (THCA-C₁), and delta-9-tetrahydrocannabiorcol (THC-C₁). Non-limiting examples of compounds comprising the delta-8-tetrahydrocannabinol class suitable for use herein can include delta-8-tetrahydrocannabinolic acid (Δ⁸-THCA) and delta-8-tetrahydrocannabinol (Δ⁸-THC).

In some embodiments, compositions disclosed herein may encompass at least one cannabinoid in the cannabicyclol class. Non-limiting examples of compounds comprising the cannabicyclol class suitable for use herein can include cannabicyclolic acid (CBLA), cannabicyclol (CBL), and cannabicyclovarin (CBLV).

In some embodiments, compositions disclosed herein may encompass at least one cannabinoid in the cannabielsoin class. Non-limiting examples of compounds comprising the cannabielsoin class suitable for use herein can include cannabielsoic acid A (CBEA-A), cannabielsoic acid B (CBEA-B), and cannabielsoin (CBE).

In some embodiments, compositions disclosed herein may encompass at least one cannabinoid in the cannabinol class. Non-limiting examples of compounds comprising the cannabinol class suitable for use herein can include cannabinolic acid (CBNA), cannabinol (CBN), cannabinol methylether (CBNM), cannabinol-C4 (CBN-C₄), cannabivarin (CBV), cannabinol-C2 (CBN-C₂), cannabiorocol (CBN-C₁), cannabinodiol (CBND), and cannabinodivarin (CBVD).

In some embodiments, compositions disclosed herein may encompass at least one cannabinoid in the cannabitriol class. Non-limiting examples of compounds comprising the cannabitriol class suitable for use herein can include cannabitriol (CBT), 10-ethoxy-9-hydroxy-delta-6a-tetrahydrocannabinol, 8,9-dihydroxy-delta-6a-tetrahydrocannabinol, cannabitriolvarin (CBTV), and ethoxy-cannabitriolvarin (CBTVE).

In some embodiments, compositions disclosed herein may encompass at least one cannabinoid not in a class. Non-limiting examples of compounds comprising cannabinoid not in a class suitable for use herein can include dehydrocannabifuran, cannabifuran, cannabichromanon, cannabicitran, 10-oxo-delta-6a-tetrahydrocannabinol, delta-9-cis-tetrahydrocannabinol, 3,4,5,6-tetrahydro-7-hydroxy-alpha-alpha-2-trimethyl-9-n-propyl-2,6-methano-2H-1-benzoxocin-5-menthanol, cannabiripsol, and trihydroxy-delta-9-tetrahydrocannabinol.

In some embodiments, compositions disclosed herein may encompass at least one cannabinoid in the cannabidiol class, tetrahydrocannabinol class, or a combination thereof. In some embodiments, compositions disclosed herein may encompass at least one of tetrahydrocannabinol (THC), cannabidiol (CBD), cannibichromene (CBC), cannabigerol (CBG), cannabinol (CBN), cannabichromevarin (CBCV), cannabichromevarin (CBCV), cannabidiphorol (CBDP), cannabielsoin (CBE), cannabigerol (CBG), Cannabicyclol (CBL), Cannabinol (CBN), cannabicitran (CBT), cannabigerovarin (CBGV), cannabigerol monomethyl ether (CBGM), Cannabivarin (CBV), delta-8-tetrahydrocannabinol (delta-8-THC, Δ8-THC), (−)-trans-Δ9-tetrahydrocannabiphorol (Δ9-THCP, (C7)-Δ9-THC, and THC-Heptyl), Δ9-tetrahydrocannabiorcol (Δ9-THCC, (C1)-Δ9-THC), tetrahydrocannabivarin (THCV, THV), dimethylheptylpyran, parahexyl, or any combination thereof. In some embodiments, compositions disclosed herein may encompass cannabidiol (CBD), tetrahydrocannabinol (THC), or a combination thereof.

In some embodiments, compositions disclosed herein may encompass CBD in a concentration of about 1 mg/mL to about 20 mg/mL (e.g., about 1, 2, 3, 4, 5, 6, 7, 8, 9, 10, 11, 12, 13, 14, 15, 16, 17, 18, 19, 20 mg/mL).

In some embodiments, compositions disclosed herein may encompass THC in a concentration of about 1 mg/mL to about 20 mg/ml (e.g., about 1, 2, 3, 4, 5, 6, 7, 8, 9, 10, 11, 12, 13, 14, 15, 16, 17, 18, 19, 20 mg/mL).

In some embodiments, compositions disclosed herein may encompass a total amount of two or more cannabinoids (e.g., CBD and THC) at a concentration of about 1 mg/mL to about 20 mg/mL (e.g., about 1, 2, 3, 4, 5, 6, 7, 8, 9, 10, 11, 12, 13, 14, 15, 16, 17, 18, 19, 20 mg/mL).

In some embodiments, compositions disclosed herein may comprise CBD, THC, or both having about 85% to 99% (e.g., about 85%, 90%, 95%, 96%, 97%, 98%, 99%) purity. In some embodiments, compositions disclosed herein may comprise pure CBD, pure THC, or both

### (b) Compositions

In some embodiments, compositions herein may encompass at least one cannabinoid disclosed herein and at least one pharmaceutically acceptable carrier or diluent. In some embodiments, compositions or solutions herein may further include various emulsifiers, surfactants, solubilizers, stabilizers, flavors, and other pharmaceutically acceptable carriers suitable for delivery to the respiratory system. In accordance with these embodiments, pharmaceutically acceptable carriers and/or diluents for use in compositions herein diluent can be a liquid carrier or diluent comprising at least one of water, propylene glycol and pharmaceutically acceptable fluids. Pharmaceutically acceptable fluids for use herein can include, but are not limited to, polar solvents, including, but not limited to, compounds that contain hydroxyl groups or other polar groups. Solvents for use herein can include, but are not limited to, water or alcohols, such as ethanol, isopropanol, and glycols including propylene glycol, polyethylene glycol, polypropylene glycol, glycol ether, glycerol and polyoxyethylene alcohols. Polar solvents can also include protic solvents, including, but not limited to, water, aqueous saline solutions with one or more pharmaceutically acceptable salt(s), alcohols, glycols or a mixture there of. In some embodiments, water for use in the present compositions can meet or exceed the applicable regulatory requirements for use in inhaled drugs.

In some embodiments, compositions herein may be a solution. In some embodiments, compositions herein may be an organic solution or an aqueous solution. In some embodiments, compositions herein may be a solution for inhalation by a subject. In some embodiments, compositions herein may be a solution for delivery of at least one cannabinoid to the respiratory system of a subject. In some embodiments, compositions herein may be a solution for intranasal administration. In some embodiments, compositions herein may not be a suspension.

In some embodiments, compositions herein may be a pharmaceutical composition for use in treating and/or preventing epilepsy, multiple sclerosis (MS), post-traumatic stress disorder (PTSD), schizophrenia, fibromyalgia, Alzheimer's disease, dementia, Parkinson's disease, Crohn's disease, glaucoma, cancer, HIV/AIDS, substance use disorder, and the like. In some embodiments, compositions herein may be a pharmaceutical composition for use in treating and/or preventing one or more symptoms associated with epilepsy, multiple sclerosis (MS), post-traumatic stress disorder (PTSD), schizophrenia, fibromyalgia, Alzheimer's disease, dementia, Parkinson's disease, Crohn's disease, glaucoma, cancer, HIV/AIDS, substance use disorder, and the like. Non-limiting examples of such symptoms include nausea, vomiting, loss of appetite, pain, insomnia, migraine, muscle spasm, seizure, anxiety, and the like.

In some embodiments, compositions herein may include propylene glycol. In some embodiments, compositions herein may include synthetic propylene glycol. In some embodiments, compositions herein may include a propylene glycol that meets food grade specifications. In some embodiments, compositions herein may include a propylene glycol having at least a MQ 400 quality level. As used herein, a propylene glycol having a MQ 400 quality level is suitable for use in critical products and applications driven by high expectations and requiring verified process control or manufacturing control (e.g., ISO 9001). In some embodiments, compositions herein may include a propylene glycol is Food Chemicals Code (FCC) and/or Food Grade (FG). In some embodiments, compositions herein may include at least about 0.1 g/mL of propylene glycol. In some embodiments, compositions herein may include about 0.1 g/mL to about 0.3 g/mL of propylene glycol (e.g., about 0.1, 0.15, 0.2, 0.25, 0.3 g/mL propylene glycol). In some embodiments, compositions herein may include about 0.17 g/mL to about 0.24 g/mL of propylene glycol (e.g., about 0.17, 0.18, 0.19, 0.20, 0.21, 0.22, 0.23, 0.24 g/mL propylene glycol).

In some embodiments, compositions herein may include glycerol. In some embodiments, compositions herein may include natural glycerol. In some embodiments, compositions herein may include a glycerol that meets food grade specifications. In some embodiments, compositions herein may include glycerol having a purity of about 99% to about 101% (e.g., about 99%, 99.5%, 100%, 100.5%, 101%). In some embodiments, compositions herein may include glycerin. As used herein, “glycerin” refers to the commercial term for a sample containing more than about 95% glycerol. In some embodiments, compositions herein may include glycerin wherein the glycerin may have a glycerol concentration of at least 95%. In some embodiments, compositions herein may include glycerin wherein the glycerin may have a glycerol concentration of at about 95% to about 99.5% (e.g., about 95%, 96%, 97%, 98%, 99%, 99.5%). In some embodiments, compositions herein may include at least about 35 mg/mL of glycerol. In some embodiments, compositions herein may include about 35 mg/mL to about 50 mg/mL of glycerol (e.g., about 35, 40, 45, 50 mg/mL glycerol). In some embodiments, compositions herein may include about 40 mg/mL to about 50 mg/ml of glycerol (e.g., about 40, 41, 42, 43, 44, 45, 46, 47, 48, 49, 90 mg/mL glycerol).

In some embodiments, compositions herein may include ethanol. The terms “ethanol” and “ethyl alcohol” are understood to have the same meaning and are used interchangeably herein. In some embodiments, compositions herein may include at least about 0.2 g/mL of ethanol. In some embodiments, compositions herein may include about 0.2 g/mL to about 0.4 g/mL of ethanol (e.g., about 0.2, 0.25, 0.3, 0.35, 0.4 g/mL ethanol). In some embodiments, compositions herein may include about 0.24 g/mL to about 0.33 g/mL of ethanol (e.g., about 0.24, 0.25, 0.26, 0.27, 0.28, 0.29, 0.30, 0.31, 0.32, 0.33 g/mL ethanol). In some embodiments, compositions herein may include ethanol having a proof of about 180 to about 200 (e.g., about 180, 185, 190, 195, 200). Proof as used herein is defined as twice the alcohol (ethanol) content by volume. As an example, an ethanol having 180 proof comprises about 90% ethanol whereas an ethanol having 200 proof comprises about 100% (i.e., ≥99.5%) ethanol. In some embodiments, compositions herein may include pure ethanol (i.e., 200 proof ethanol). In some embodiments, compositions herein may include ethanol having a proof of about 200.

In some embodiments, compositions herein may include water. In some embodiments, compositions herein may include at least about 0.3 g/ml of water. In some embodiments, compositions herein may include about 0.3 g/mL to about 0.6 g/mL water (e.g., about 0.3, 0.35, 0.4, 0.45, 0.5, 0.55, 0.6 g/mL water). In some embodiments, compositions herein may include deionized water. In some embodiments, compositions herein may include distilled water. In some embodiments, compositions herein may include Type I water. Type I water is defined by the American Society for Testing and Materials (ASTM) as having a resistivity of >18 MΩ-cm, a conductivity of <0.056 μS/cm and <50 ppb of Total Organic Carbons (TOC).

In some embodiments, compositions herein may include one or more cannabinoids, water, ethanol, and propylene glycol. The cannabinoid may be present in a concentration of about 1 mg/mL to about 20 mg/mL, the propylene glycol present in a concentration of about 0.1 g/mL to about 0.3 g/mL; and the ethanol present in a concentration of about 0.2 g/mL to about 0.4 g/mL. In some embodiments, the water is present water is present in a concentration of about 0.3 g/mL to about 0.6 g/mL. In some embodiments, the composition may further comprise glycerol. The glycerol may be present in a concentration of about 35 mg/mL to about 50 mg/mL.

In some embodiments, compositions herein may have a surface tension of at least about 20 mN/m (millinewtons/meter). In some embodiments, compositions herein may have a surface tension of about 20 mN/m to about 35 mN/m (e.g., about 20, 25, 30, 35 mN/m). In some embodiments, compositions herein may have a surface tension of about 25 mN/m to about 34 mN/m (e.g., about 25, 26, 27, 28, 29, 30, 31, 32, 33, 34 mN/m). In some embodiments, compositions herein may have a surface tension of about 31 mN/m to about 33 mN/m (e.g., about 31.0, 31.25, 31.5, 31.75, 32.0, 32.25, 32.5, 32.75, 33.0 mN/m). In some embodiments, compositions herein may have a surface tension suitable for use with a droplet delivery device disclosed herein.

### II. Methods

Effective and efficient delivery of substances to the respiratory system of a user, and the synchronization of the administration of substances to the respiratory system of the user with the inspiration/expiration cycle of the user has always posed a problem. For instance, optimum deposition in alveolar airways generally requires droplets with aerodynamic diameters in the ranges of 1 to 6 μm, with droplets below about 4 μm shown to more effectively reach the alveolar region of the lungs and larger droplets above about 6 μm shown to typically deposited on the tongue or strike the throat and coat the bronchial passages. Smaller droplets, for example less than about 1 μm, penetrate more deeply into the lungs and have a tendency to be exhaled. As such, methods for delivering a fluid composition comprising at least one cannabinoid as an ejected stream of droplets in a respirable range in accordance with aspects of the disclosure requires the ability to precisely target droplet sizes for the particular use.

In some embodiments, methods of making any one of the compositions herein can include adding at least one cannabinoid to at least one pharmaceutically acceptable carrier or diluent. In some embodiments, methods of making any one of the compositions herein can include adding propylene glycol disclosed herein to at least one cannabinoid disclosed herein to form a propylene glycol/cannabinoid mixture. In some embodiments, methods of making any one of the compositions herein can include adding propylene glycol disclosed herein to at least one cannabinoid disclosed herein until the cannabinoid is at least about 95% (e.g., about 95%, 96%, 97%, 98%, 99%, 99.5%, 100%) dissolved in the propylene glycol to form a propylene glycol/cannabinoid mixture. In some embodiments, an ethanol disclosed herein can be added to the propylene glycol/cannabinoid solution to form an ethanol/propylene glycol/cannabinoid mixture. In some embodiments, methods of making any one of the compositions herein can include adding ethanol disclosed herein to a propylene glycol/cannabinoid mixture until the cannabinoid is at least about 95% (e.g., about 95%, 96%, 97%, 98%, 99%, 99.5%, 100%) dissolved to form an ethanol/propylene glycol/cannabinoid mixture solution. In some embodiments, water disclosed herein can be added to the ethanol/propylene glycol/cannabinoid mixture solution to form a composition of the present disclosure. In some embodiments, water can be added to the ethanol/propylene glycol/cannabinoid mixture solution to form a composition of the present disclosure after cannabinoid(s) are at least about 95% (e.g., about 95%, 96%, 97%, 98%, 99%, 99.5% 100%) dissolved in the ethanol/propylene glycol/cannabinoid mixture solution. In some embodiments, water can be added to the ethanol/propylene glycol/cannabinoid mixture solution to form a composition of the present disclosure after cannabinoid(s) are completely dissolved in the ethanol/propylene glycol/cannabinoid mixture solution. In some embodiments, water disclosed herein can be added to the ethanol/propylene glycol/cannabinoid mixture solution in increments while stirring to form a composition of the present disclosure. In some embodiments, water disclosed herein can be added to the ethanol/propylene glycol/cannabinoid mixture solution in about 200 μL to about 500 μL (e.g., about 200, 250, 300, 350, 400, 450, 500 μL) increments while stirring to form a composition of the present disclosure. In some embodiments, methods of making any one of the compositions herein can be performed at temperatures equal to or higher than about 20° C. In some embodiments, methods of making any one of the compositions herein can be performed at temperatures ranging from about 0° C. to about 100° C. In some embodiments, methods of making any one of the compositions herein can be performed at room temperature (i.e., about 25° C.±5° C.).

In certain embodiments, the present disclosure provides methods for delivering a fluid composition comprising at least one cannabinoid as an ejected stream of droplets in a respirable range in accordance with aspects of the disclosure requires the ability to precisely target droplet sizes for the particular use. In accordance with certain aspects of the disclosure, effective deposition of an ejected stream of droplets of a fluid composition comprising at least one cannabinoid into the lungs of a user generally requires droplets less than about 5-6 μm, e.g., less than about 3.2 μm, in diameter. Without intending to be limited by theory, to deliver an ejected stream of droplets to the lungs, a droplet delivery device must impart a momentum that is sufficiently high to permit ejection out of the device, but sufficiently low to prevent deposition on the tongue or in the back of the throat. Droplets below approximately 5-6 μm in diameter are transported almost completely by motion of the airstream and entrained air that carry them and not by their own momentum.

In certain embodiments, the methods of the present disclosure result in minimal or no mouth or throat irritation. In certain embodiments, the methods include generating an ejected stream of droplets of a fluid composition comprising at least one cannabinoid with coordinated and precise timing during a user's inspiration cycle to as to maximize delivery into the respiratory system, while minimizing or eliminating mouth or throat irritation. Without intending to be limited by theory, as described herein, the small droplets generated via the methods of the disclosure are transported almost completely by motion of airstream and entrained air. Using this entrained motion and tuned droplet size, the ejection of droplets may be focused so as to eject during peak flow of the inspiration cycle so as to optimize inhalation into the target site in the respiratory system (e.g., deep lungs), while minimizing inadvertent delivery to non-desired sites in the respiratory system (e.g., mouth and throat).

As discussed above, effective delivery of droplets deep into the lung airways require droplets that are less than about 5-6 microns in diameter, specifically droplets with mass mean aerodynamic diameters (MMAD) that are less than about 5 microns. However, for certain agents and uses, droplets about 1 μm or smaller for quick adsorption in the deep lung may be desirable, e.g., it may be desired to utilize droplets less than 4 μm, less than 3.2 μm, less than 3 μm, less than 2 μm, and less than 1 μm for the delivery at least one cannabinoid to the deep lungs. The mass mean aerodynamic diameter is defined as the diameter at which 50% of the droplets by mass are larger and 50% are smaller. In certain aspects of the disclosure, in order to deposit in the alveolar airways, droplets in this size range must have momentum that is sufficiently high to permit ejection out of the droplet delivery device, but sufficiently low to overcome deposition onto the tongue (soft palate) or pharynx.

In certain embodiments, methods for generating an ejected stream of droplets from a fluid composition comprising at least one cannabinoid for delivery to the respiratory system of user are provided. In certain embodiments, the ejected stream of droplets is generated in a controllable and defined droplet size range. By way of example, the droplet size range includes at least about 50%, at least about 60%, at least about 70%, at least about 85%, at least about 90%, between about 50% and about 90%, between about 60% and about 90%, between about 70% and about 90%, etc., of the ejected droplets are in the respirable range of below about 5 μm, below about 4 μm, below about 3.7 μm, below about 3.5 μm, below about 3.2 μm, below about 3.0 μm, below about 2 μm, between about 0.7 μm and about 4 μm, between about 0.7 μm and about 3.2 μm, between about 0.7 μm and about 3 μm, between about 0.7 μm and about 2.5 μm, between about 0.7 μm and about 2.0 μm, between about 0.7 μm and about 1.5 μm, between about 0.7 μm and about 1.0 μm, etc.

In other embodiments, the ejected stream of droplets may have one or more diameters, such that droplets having multiple diameters are generated so as to target multiple regions in the airways (mouth, tongue, throat, upper airways, lower airways, deep lung, etc.) By way of example, droplet diameters may range from about 0.7 μm to about 200 μm, about 0.7 μm to about 100 μm, about 0.7 μm to about 60 μm, about 0.7 μm to about 40 μm, about 0.7 μm to about 20 μm, about 0.7 μm to about 5 μm, about 0.7 μm to about 4.7 μm, about 0.7 μm to about 4 μm, about 0.7 μm to about 3.0 μm, about 0.7 μm to about 2.5 μm, about 0.7 μm and about 2.0 μm, about 0.7 μm and about 1.5 μm, about 0.7 μm and about 1.0 μm, about 5 μm to about 20 μm, about 5 μm to about 10 μm, and combinations thereof. In particular embodiments, at least a fraction of the droplets has diameters in the respirable range, while other droplets may have diameters in other sizes so as to target non-respirable locations (e.g., larger than about 5 μm). Illustrative ejected droplet streams in this regard might have 50%-70% of droplets in the respirable range (less than about 5 μm), and 30%-50% outside of the respirable range (about 5 μm-about 10 μm, about 5 μm-about 20 μm, etc.)

In some embodiments embodiment, methods for delivering safe, suitable, and repeatable dosages of a fluid composition comprising at least one cannabinoid to the respiratory system of a user are provided herein. The methods of such embodiments may deliver an ejected stream of droplets to the desired location within the respiratory system of the user. In certain embodiments, the methods may be capable of delivering a defined volume of fluid in the form of an ejected stream of droplets such that an adequate and repeatable high percentage of the droplets are delivered into the desired location within the airways, e.g., the alveolar airways of the user during use.

In some embodiments, methods herein may include delivering a fluid composition comprising at least one cannabinoid as an ejected stream of droplets in a respirable range to the respiratory system of a user. In certain embodiments, the method comprises (a) generating an ejected stream of droplets from the fluid composition via a droplet delivery device, wherein at least about 50% of the ejected stream of droplets have an average ejected droplet diameter of less than about 6 μm; and (b) delivering the ejected stream of droplets to the respiratory system of the user such that at least about 50% of the mass of the ejected stream of droplets is delivered in a respirable range to the respiratory system of a user during use.

In certain embodiments, the methods of the disclosure may be used to treat various diseases, disorders and conditions, promote or regulate various physiological activities, and combinations thereof, by delivering a fluid composition comprising at least one cannabinoid to the respiratory system of a user. In this regard, the methods of the disclosure may be used to deliver at least one cannabinoid locally to the respiratory system, and/or systemically to the body. In certain embodiments, the at least one cannabinoid is delivered to a user to treat or ameliorate one or more diseases, conditions or disorders selected from epilepsy, multiple sclerosis (MS), post-traumatic stress disorder (PTSD), schizophrenia, fibromyalgia, Alzheimer's disease, dementia, Parkinson's disease, Crohn's disease, glaucoma, cancer, HIV/AIDS, and/or substance use disorder. In other embodiments, the at least one cannabinoid is delivered to a user to ameliorate and/or reduce incidence of nausea, vomiting, loss of appetite, pain, insomnia, migraine, muscle spasm, seizure, and/or anxiety.

In accordance with the disclosure, any suitable droplet delivery device may be used in connection with the the disclosure. By way of example, droplet delivery devices that may be used with the methods described herein include, but are not limited to, those described in PCT/US2017/030913 (WO2017/192767), PCT/2017/030917 (WO2017/192771), PCT/2017/030919 (WO2017/192773), PCT/US2017/030921 (WO2017/192774), PCT/US2017/030929 (WO2017/192782), PCT/2017/030925 (WO2017/192778), PCT/US2018/054417 (WO 2019/071008), PCT/US2018/056300 (WO 2019/079461, PCT/2018/059874 (WO2019/094628), PCT/2019/012691 (WO2019/136437), PCT/US2019/25321 (WO2019/195239), PCT/2019/054042 (WO2020/072478), PCT/US2020/014785 (WO2020/154497), PCT/US2020/032383 (WO2020/227717), PCT/US2020/040132 (WO2020/264501), PCT/US2022/035492, PCT/US2022/026176, PCT/US2022/034552, U.S. patent application Ser. No. 17/846,902, U.S. Provisional Patent Application No. 63/256,245, U.S. Provisional Patent Application No. 63/318,202, U.S. Provisional Patent Application No. 63/323,770, U.S. Provisional Patent Application No. 63/346,794, U.S. Provisional Patent Application No. 63/337,885, U.S. Provisional Patent Application No. 63/390,170, U.S. Provisional Patent Application No. 63/390,209, and U.S. Provisional Patent Application No. 63/390,228, the disclosures of which are each incorporated herein by reference in their entirety.

In certain embodiments, compositions disclosed herein may be used with a “push mode” droplet delivery device that preferably does not include a heating requirement that could result in undesirable byproducts and comprises: a container assembly with an mouthpiece port; a reservoir disposed within or in fluid communication with the container assembly to supply a volume of fluid of the composition, an ejector bracket in fluid communication with the reservoir, the ejector bracket including a mesh with a membrane operably coupled to an electronic transducer (such as am ultrasonic transducer preferably including piezoelectric material) with the membrane between the transducer and the mesh, wherein the mesh includes a plurality of openings formed through the mesh's thickness, and wherein the transducer is coupled to a power source and is operable to oscillate the membrane and generate an ejected stream of droplets of composition through the mesh, and an ejection channel within the container assembly configured to direct the ejected stream of droplets from the mesh to the outlet. The vibrating membrane “pushing” liquid composition through the mesh is referred to as “push mode” ejection and devices in embodiments of the push mode invention may be referred to as push mode devices. A non-limiting example of such a device is described in U.S. patent application Ser. No. 17/846,902 and PCT/US2022/034552, the disclosures of which are each incorporated herein by reference in their entirety.

In one embodiment, the droplet delivery device may be configured to provide for ejection of droplets after a breath initiation period, e.g., 0.1-0.5 seconds. The device may be configured to sense the initiation of the inspiration cycles, allowing a short period of time, e.g., 0.1-0.5 seconds as to form a steady inspiration flow. Once the device senses a steady inspiration flow, the device may activate an ejector mechanism to initiate ejection of the small droplets for inhalation into the target site of the respiratory system. Optionally, the device may control the ejector mechanism to discontinue generation of droplets at a specified end portion of the inspiration cycle, so as to allow for complete inhalation of the droplets to the target site of the respiratory system. Such a device provides for an improved method of delivering droplets to the respiratory system of a user with minimal or no mouth or throat irritation.

In certain embodiments, methods herein including generating an ejected stream of droplets from a fluid composition comprising at least one cannabinoid via an droplet delivery device comprising an ejector mechanism having an aperture plate (or mesh), the aperture plate (or mesh) having a plurality of openings formed through its thickness wherein the droplet delivery device is configured to directly or indirectly oscillate the aperture plate (or mesh) at a frequency to thereby generate the ejected stream of droplets, wherein at least about 50% of the ejected stream of droplets have an average ejected droplet diameter of less than about 6 μm; and delivering the ejected stream of droplets to the respiratory system of the user such that at least about 50% of the mass of the ejected stream of droplets is delivered in a respirable range to the respiratory system of a user during use.

In certain embodiments, the ejected stream of droplets of a fluid composition comprising at least one cannabinoid herein may be generated via an ejector mechanism configured to provide coordinated and precise control of droplet size. In certain embodiments, the ejector mechanism of the droplet delivery device may comprise at least one aperture plate (or mesh) with a plurality of openings formed through its thickness for ejecting droplets, wherein at least one surface of the aperture plate (or mesh) is configured to provide a desired surface contact angle. In certain embodiments, the aperture plate (or mesh) may be configured such that at least one surface is configured with a desired surface contact angle to facilitate generation of droplets with the desired droplet size distribution, e.g., less than 4 μm, less than about 3.2 microns, less than about 3 microns, less than about 2 microns, less than about 1.5 microns, less than about 1 micron, etc.

In certain embodiments, the aperture plate (or mesh) has a plurality of openings formed through its thickness and at least the fluid entrance side of one or more of said plurality of openings configured so as to provide a surface contact angle of less than 90 degrees. In certain embodiments, at least about 50% of the droplets have an average ejected droplet diameter of less than about 6 microns during use. In some embodiments, at least a portion of the interior of at least one of the openings near the fluid entrance side is configured so as to provide a surface contact angle of less than 90 degree.

In other embodiments, the aperture plate (or mesh) is configured such that at least the fluid exit side of one or more of said plurality of openings is configured to provide a surface contact angle of greater than 90 degrees. In some embodiments, at least a portion of the interior of at least one of the openings near the fluid exit side is configured so as to provide a surface contact angle of greater than 90 degrees.

In certain embodiments, at least the fluid entrance surface of one or more openings of the aperture plate (or mesh) and the fluid exit surface of one or more openings of the aperture plate (or mesh) are configured (e.g., treated, coated, surface modified, or a combination thereof) to provide a desired surface contact angle. In some embodiments, at least a portion of the interior of at least one of the openings near the fluid entrance side is configured so as to provide a desired surface contact angle. By way of example, the fluid entrance surface and/or interior surface of one or more openings of the aperture plate (or mesh) may be configured to have a surface contact angle of less than about 80 degrees, less than about 70 degrees, less than about 50 degrees, less than about 55 degrees, less than about 50 degrees, less than about 40 degrees, less than about 35 degrees, less than about 30 degrees, less than about 20 degrees, less than about 10 degrees, between about 10 degrees and about 80 degrees, between about 10 degrees and about 60 degrees, between about 20 degrees and about 55 degrees, between about 10 and about 35 degrees, between about 15 and about 35 degrees, etc. By way of a further example, the fluid exit surface and/or interior surface of one or more openings of the aperture plate (or mesh) may be configured to have a surface contact angle of greater than greater than 90 degrees, between 90 degrees and 140 degrees, between 90 degrees and 135 degrees, between 100 degrees and 140 degrees, between 100 degrees and 135 degrees, between 90 degrees and 110 degrees, etc.

In certain aspects, the droplet delivery device is capable of delivering a defined volume of fluid (fixed dose) in the form of an ejected stream of droplets having a small average ejected diameter such that an adequate and repeatable high percentage of the droplets are delivered into the desired location within the airways, e.g., the alveolar airways of the user during use. In certain embodiments, the average droplet diameters may range from about 0.7 μm to about 5 μm, about 0.7 μm to about 4.7 μm, about 0.7 μm to about 4 μm, about 0.7 μm to about 3.2 μm, about 0.7 μm to about 2.5 μm, about 0.7 μm to about 1.3 μm, etc. In certain embodiments, the average droplet diameters may be less than about 4 microns, less than about 3.2 microns, less than about 3 microns, less than about 2 microns, less than about 1.5 microns, less than about 1 micron, etc. In certain embodiments, the average droplet diameters may range from about 1 μm to about 2 μm (e.g., about 1.0, 1.1, 1.2, 1.3, 1.4, 1.5, 1.6, 1.7, 1.8, 1.9, 2.0 μm). In certain embodiments, the average droplet diameters may range from about 3 μm to about 4 μm (e.g., about 3.0, 3.1, 3.2, 3.3, 3.4, 3.5, 3.6, 3.7, 3.8, 3.9, 4.0 μm). One of skill in the art can appreciate that the average droplet diameters may be optimized to meet clinical need while staying within an acceptable respirable range.

In certain embodiments, one or more surfaces of the aperture plate (or mesh) may be modified, treated, coated, or a combination thereof to achieve the desired surface contact angle. In certain aspects, the one or more surfaces of the aperture plate (or mesh) may be modified, treated, coated, or a combination thereof so as to affect surface hydrophobicity. By way of examples, one or more surfaces of the aperture plate (or mesh) may be modified, treated, coated, or a combination thereof so as to result in at least one more hydrophilic surface on the aperture plate (or mesh), optionally at least one more hydrophobic surface on the aperture plate (or mesh), or a combination thereof. In certain embodiments, at least the fluid entrance side, and optionally the fluid exit surface side are configured so as to have a desired surface contact angle. In certain embodiments, at least a portion of the interior surface of one or more openings may be configured so as to have a desired surface contact angle.

In addition to aperture plate (or mesh) surface contact angle, several features of the ejector mechanism allow for precise dosing of specific droplet sizes. For instance, droplet size is set, in part, by the diameter of the openings in the aperture plate (or mesh), which are formed with high accuracy. By way of example, the openings in the aperture plate (or mesh) at the fluid exit side of the aperture plate (or mesh) may range in size from 1 μm to 6 μm, from 2 μm to 5 μm, from 3 μm to 5 μm, from 3 μm to 4 μm, about 1.7 μm, about 2.0 μm, about 3.5 μm, about 3.9 μm, etc. In certain embodiments, the aperture plate (or mesh) may include openings having different cross-sectional shapes or diameters to thereby provide ejected droplets having different average ejected droplet diameters. Ejector rate also influences droplet size. Ejection rate, in droplets per second, is fixed by the frequency of the aperture plate (or mesh) vibration, e.g., 108-kHz, etc.

In certain aspects of the disclosure, desired surface contact angles may be formed by creating hydrophilic surfaces, e.g., through treating, coating, surface modifying, or a combination thereof. A surface is considered to be hydrophilic when that angle is less than about 80 degrees, about 70 degrees, about 60 degrees, about 55 degrees, about 50 degrees, etc., and may be considered to be super hydrophilic when that angle is less than about 10 to 20 degrees (droplet tends to spread out across the surface). The strength of the hydrophilic effect may be measured by the angle between the edge of a droplet of water and the surface of the aperture plate (or mesh).

By way of example, the aperture plate (or mesh) can be formed of a metal, e.g., stainless steel, nickel, cobalt, titanium, iridium, platinum, or palladium or alloys thereof, and configured to achieve the desired contact angles as described herein. Alternatively, the aperture plate (or mesh) can be formed of suitable polymeric material and be configured to achieve the desired surface contact angles, as described herein. By way of example, the aperture plate (or mesh) may be composed of a material selected from the group consisting of polyethylene naphthalate (PEN), polyetheretherketone (PEEK), polyimide, polyetherimide, polyvinylidine fluoride (PVDF), ultra-high molecular weight polyethylene (UHMWPE), polysulfone, nickel, nickel-cobalt, nickel-palladium, palladium, platinum, metal alloys thereof, and combinations thereof. Further, in certain aspects, the aperture plate (or mesh) may comprise a domed shape.

By way of example, the desired surface contact angle may be created on a surface of an aperture plate (or mesh) by increasing the surface energy through creation of a polar surface. Exemplary methods to increase surface energy comprise forming an oxide surface on a metallic ejector aperture plate (or mesh) which is polar. In accordance with aspects of the disclosure, exemplary methods for creating a hydrophilic surface contact angle on an aperture plate (or mesh) including dip coating methods, etching methods, and chemical deposition methods. Dip coating methods comprise dipping the aperture plate (or mesh) into a solution comprising a desired coating and a solvent, which solution will form a hydrophilic coating on the surface when the solvent evaporates. Chemical deposition methods include known deposition methods, e.g., plasma etch, plasma coating, plasma deposition, CVD, electroless plating, electroplating, etc., wherein the chemical deposition uses a plasma or vapor to open the bonds on the surface of the aperture plate (or mesh) so that oxygen or hydroxyl molecules attach to the surface rendering it polar. Etching methods include non-chemical etching methods using surface roughening.

In certain embodiments, any deposited hydrophilic layer is significantly thinner than the opening size such that it does not impact the size of the generated droplets. In certain embodiments, the surface treatment may extend into at least a portion of one or more openings of the aperture plate (or mesh) so as to form a hydrophilic surface within at least a portion of one or more openings.

In certain embodiments, the desired surface contact angle may be obtained through surface roughening achieved, e.g., via non-chemical etching. Without intending to be limited by theory, as an approximation, the Wenzel Contact Angle equation, “Apparent Contact Angles on Rough Surfaces: the Wenzel Contact Angle Revisited”, Wolansky and Marmur, Colloids and Surfaces A, 156 (1999) pp. 381-388, may be used to estimate surface contact angle. The Wenzel equation yields contact angles for liquid drops on rough surfaces. It assumes no hysteresis in the contact angle, and this is an approximation.

In certain embodiments, the aperture plate (or mesh) may optionally be surface sputtered with a thin layer (e.g., about 30 to about 150 nm, about 60 nm to about 100 nm, about 30 nm, about 60 nm, about 80 nm, about 100 nm, etc. thick sputtering) of a precious metal, such as gold (Au), palladium (Pd), platinum (Pt), silver (Ag) and precious metal alloys. In certain embodiments, the surface may be sputtered with a thin layer of palladium. The precious metal layer may then be etched at varying etch powers, e.g., low, medium or high etching power to provide a desired surface contact angle. To provide the desired contact angle, the etch may be performed once, twice, three times, four times, etc.

In other embodiments, the aperture plate (or mesh) may be coated on at least the fluid entrance side of the aperture plate (or mesh) with a hydrophilic polymer to achieve the desired surface contact angle. In yet other embodiments, the aperture plate (or mesh) may be coated on at least a portion of the interior surface of one or more openings, within the entire interior surface of one or more openings, on both the fluid entrance side and the fluid ejection surface of the aperture plate (or mesh), and combinations thereof. Any known hydrophilic polymer suitable for use in medical applications may be used.

Any suitable hydrophilic coating to achieve the desired surface contact angle on the fluid entrance side of the ejector aperture plate (or mesh) may be used. Exemplary hydrophilic coating materials include, but are not limited to siloxane based coatings, isocyante based coatings, ethylene oxide based coatings, polyisocyanate based coatings, hydrocyclosiloxane based coatings, hydroxyalkylmethacrylate based coatings, hydroxyalkylacrylate based coatings, glycidylmethacrylate based coatings, propylene oxide based coatings, N-vinyl-2-pyrrolidone based coatings, latex based coatings, polyvinylchloride based coatings, polyurethane based coatings, etc.

By way of non-limiting example, a suitable hydrophilic coating may comprise a single layer hydrophilic surface formed by a process of cleaning the intended surface with a low pressure plasma and then dipping the surface into a solution of organophosphorous acids which self-assemble into a polar monolayer (e.g., see Aculon U.S. Pat. No. 8,658,258A, which is incorporated herein by reference). These layers are typically less than 10 nm thick, which is significantly less than a micron-sized hole. Contact angles as low as 10 degrees can be achieved using such coatings.

In other embodiments, the aperture plate (or mesh) may optionally be coated on the fluid exit side with a hydrophobic coating. Any known hydrophobic polymer suitable for use in medical applications may be used, e.g., polytetrafluoroethylene (Teflon), siloxane based coatings, paraffin, polyisobutylene, polysulfone, etc. The surface of the hydrophobic coating may be chemically or structurally modified or treated to further enhance or control the surface contact angle, as desired.

In certain embodiments, the aperture plate (or mesh) may be coated with a siloxane based coating to provide an initial hydrophobic coating, which siloxane based coating is thereafter masked or shielded in a suitable manner on the fluid exit side. Following masking, the masked aperture plate (or mesh) may thereafter be exposed to an oxidizing treatment to render the siloxane coating hydrophilic on the exposed (unmasked) portions thereof, i.e., the fluid entrance sides. In this manner, in certain embodiments of the disclosure, the same siloxane based coating may provide both hydrophilic and hydrophobic coatings to surfaces of the aperture plate (or mesh). By way of example, such siloxane coatings may be selected from siloxanes known for use in medical applications, such as 2,4,6,8-Tetramethylcyclotetrasiloxane, or 1,1,3,3-Tetramethyldisiloxane.

The aperture plate (or mesh) may be metallic or polymer with openings about the diameter of the desired droplets (as discussed further herein). By way of non-limiting example, the aperture plate (or mesh) may be formed from silicon, silicon carbide, nickel palladium, or a high stiffness polymer such as polyetheretherketone (PEEK), poly-amide, Kapton or Ultra High Molecular Weight Polyethylene (UHMWPE). When using a polymer aperture plate (or mesh), the openings may be produced by rolling, stamping, laser ablation, bulk etching or other known micro-machining processes. When using silicon and SiC for the aperture plate (or mesh), the openings may be formed using typical semiconductor processes. Without being limited, these silicon materials can be formed by bulk micro-machining processes, such as wet etching. In addition, the aperture plate (or mesh) opening area may be formed to have a dome-like shape to increase the stiffness of the aperture plate (or mesh) and to creating uniform ejection accelerations.

The aperture plate (or mesh) may have an array of opening ranging from, e.g., 100 to 10,000 openings, 500 to 10,000 openings, etc. The openings may generally have a fluid exit side diameter similar to that of the desired droplets, e.g., of 0.5 μm to 100 μm diameter, 1 μm to 20 μm, 1 μm to 10 μm, 1 μm to 5 μm, 1 μm to 4 μm, etc., as described further herein. The fluid entrance side diameter may range from between about 30 μm to 300 μm, about 75 μm to about 200 μm, about 100 μm to about 200 μm, etc. Aperture plate (or mesh)s may be formed to have a thickness of between about 100 μm to about 925 μm, between about 100 μm and about 300 μm.

As described above, the aperture plate (or mesh) may include various treatments, coatings surface modifications, or combinations thereof, on one or more surfaces thereof. For example, in certain embodiments, the aperture plate (or mesh) may include various combinations of a hydrophilic coating on one or more surfaces, an optional hydrophobic coating on one or more surfaces, native surfaces, surface etchings, etc. In one embodiment, the aperture plate (or mesh) may be non-chemically etched on the fluid entrance side of the aperture plate (or mesh) (fluid reservoir facing side), with etching, a hydrophobic coating, or no treatment on the fluid exit side. In another embodiment, the aperture plate (or mesh) may include a hydrophilic coating on at least the fluid entrance side of the aperture plate (or mesh) (fluid reservoir facing side), a hydrophilic coating within at least a portion of the interior of one or more openings, or combinations thereof. In other embodiments, the aperture plate (or mesh) may include a hydrophobic coating on the droplet exit side of the aperture plate (or mesh)—alone or in combination with one or more hydrophilic coatings. A gas or liquid process may be used to form the hydrophobic and hydrophilic surfaces. For example, hydrophilic and hydrophobic surfaces can be formed using liquid coating, sputtering, CVD, plasma deposition, ion implantation, etc.

The aperture plate (or mesh) s may be produced, e.g., by semiconductor techniques, stamping, rolling or laser ablation. Rolling may be preferred because more precise forming pressures are possible and continuous production for material from rolls allows lower-cost manufacturing. Because the material stiffness of polymers (especially the UHMWPE) is lower than metals such as stainless steel or palladium-nickel, ribs on the fluid or air side of the aperture plate (or mesh) may also be formed at the time of rolling or prior to laser ablation. Similarly, a metallic annulus may be used to stiffen the edge of the aperture plate (or mesh) against flexure. In addition, the aperture plate (or mesh) area can be formed to have a dome-like shape to increase the stiffness of the aperture plate (or mesh) and creating uniform ejection accelerations.

All publications and patent applications cited in this specification are herein incorporated by reference as if each individual publication or patent application were specifically, and individually, indicated to be incorporated by reference.

### EXAMPLES

The following examples are included to illustrate certain embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that changes can be made in some embodiments which are disclosed and still obtain a like or similar result without departing from the spirit and scope of embodiments of the inventions.

### Example 1

In one exemplary method, a formulation containing CBD was prepared. CBD used for these exemplary formulations was synthetically produced and had a 100% enantiomeric purity for (−)-Cannabidiol as assessed by HLPC. A test for impurities and residual solvents in the CBD determined that only the solvent likely to be present in the preparation was isooctane at less than 500 ppm. Accordingly, the CBD used in these exemplary formulations was in compliance with the current USP <467> residual solvent requirements.

Several formulations were prepared using concentrations detailed in Table 1. The concentrations of components in the formulations provided in Table 1 were based on the maximum potency per unit dose for respiratory (inhalation) excipients according to the FDA.

TABLE 1

| | | | | | Surface |
|---|---|---|---|---|---|
| CBD | Propylene glycol | Glycerol | ETOH | water | Tension |
| (mg/mL) | (g/mL) | (g/mL) | (g/mL) | (g/mL) | (mN/m) |
| 18.41 | 0.23 | — | 0.329 | 0.361 | 28.62 |
| 13.50 | 0.23 | — | 0.322 | 0.376 | 31.32 |
| 8.43 | 0.21 | — | 0.301 | 0.415 | 32.06 |
| 6.12 | 0.20 | — | 0.292 | 0.434 | 32.32 |
| 3.90 | 0.20 | — | 0.279 | 0.459 | 32.32 |
| 1.80 | 0.18 | — | 0.258 | 0.500 | 33.29 |
| 1.03 | 0.17 | — | 0.245 | 0.525 | 34.05 |
| 9.42 | 0.24 | 0.047 | 0.283 | 0.377 | 32.15 |
| 6.91 | 0.23 | 0.046 | 0.276 | 0.391 | 32.13 |
| 4.31 | 0.22 | 0.043 | 0.258 | 0.431 | 32.82 |
| 1.98 | 0.20 | 0.040 | 0.238 | 0.476 | 33.48 |

To prepare the formulations, first CBD was weighed out the using a microbalance in a 10 mL beaker. Propylene glycol was then added to the beaker on microbalance. For formulations that included glycerol, glycerol was added to beaker on microbalance after the addition of propylene glycol. Next, a stir bar was added to the beaker and stirring the solution was started. Ethanol was added to the beaker containing the solution using a pipettor. The solution was allowed to stir till all CBD was dissolved. Once the CBD was dissolved, water was slowly added to the solution in 250 μL increments until the desired amount was achieved. The formulation and testing was completed at room temperature.

Surface tension was determined for each formulation using a tensiometer. The

results are provided in Table 1. Surface tension plays a role in aerosol characteristics. For example, the correct surface tension is a necessary factor to achieve good particle ejection.

1. A composition comprising:
a cannabinoid in a concentration of about 1 mg/mL to about 20 mg/ml;
propylene glycol in a concentration of about 0.1 g/mL to about 0.3 g/mL;
ethanol in a concentration of about 0.2 g/mL to about 0.4 g/mL; and
water,
wherein the composition has a surface tension of about 20 mN/m to about 35 mN/m and the composition is a solution.

2. The composition of claim 1, wherein the composition has a surface tension of about 25 mN/m to about 34 mN/m.

3. The composition of claim 2, wherein the composition has a surface tension of about 31 mN/m to about 33 mN/m.

4. The composition of claim 1, wherein the composition further comprises glycerol in a concentration of about 35 mg/mL to about 50 mg/mL.

5. The composition of claim 1, wherein the composition comprises glycerin and the glycerin has a glycerol concentration of at least 95% or at least 99%.

6. The composition of claim 1, wherein the cannabinoid is selected from: tetrahydrocannabinol (THC), cannabidiol (CBD), cannibichromene (CBC), cannabigerol (CBG), cannabinol (CBN), cannabichromevarin (CBCV), cannabichromevarin (CBCV), cannabidiphorol (CBDP), cannabielsoin (CBE), cannabigerol (CBG), Cannabicyclol (CBL), Cannabinol (CBN), cannabicitran (CBT), cannabigerovarin (CBGV), cannabigerol monomethyl ether (CBGM), Cannabivarin (CBV), delta-8-tetrahydrocannabinol (delta-8-THC, Δ8-THC), (−)-trans-Δ9-tetrahydrocannabiphorol (Δ9-THCP, (C7)-Δ9-THC, and THC-Heptyl), Δ9-tetrahydrocannabiorcol (Δ9-THCC, (C1)-Δ9-THC), tetrahydrocannabivarin (THCV, THV), dimethylheptylpyran, parahexyl, or any combination thereof.

7. The composition of claim 6, wherein the cannabinoid is cannabidiol (CBD).

8. The composition of claim 6, wherein the cannabinoid is tetrahydrocannabinol (THC).

9. The composition of claim 1, wherein the cannabinoid is at least 90% pure.

10. The composition of claim 1, wherein the propylene glycol is present in an amount of about 0.15 g/mL to about 0.25 g/mL.

11. The composition of claim 1, wherein the ethanol is present in an amount of about 0.24 g/mL to about 0.33 g/mL.

12. The composition of claim 1, wherein the ethanol is about 180 to about 200 proof.

13. The composition of claim 1, wherein the water is present in an amount of about 0.3 g/mL to about 0.6 g/mL.

14. The composition of claim 1, wherein the composition is a composition for inhalation by a subject.

15. The composition of anyone of claim 1, wherein the composition is delivered to the respiratory system of a subject.

16. The composition of claim 1, wherein the composition is a pharmaceutical composition for use in treating one or more of epilepsy, multiple sclerosis (MS), post-traumatic stress disorder (PTSD), schizophrenia, fibromyalgia, Alzheimer's disease, dementia, Parkinson's disease, Crohn's disease, glaucoma, cancer, HIV/AIDS, and substance use disorder.

17. A method of making the composition of claim 1, comprising:
(a) adding the propylene glycol to the cannabinoid and stirring until combined to form a propylene glycol/cannabinoid mixture;
(b) adding the ethanol to the propylene glycol/cannabinoid mixture to form an ethanol/propylene glycol/cannabinoid mixture; and
(c) adding the water in increments to the ethanol/propylene glycol/cannabinoid mixture while stirring until the full amount of water is added,
thereby making the composition.

18. The method of claim 17, wherein the cannabinoid is dissolved before adding water in (c).

19. The method of claim 17, wherein the method is performed at temperatures equal to or higher than about 20° C.

20. A method for delivering a composition according to claim 1 as an ejected stream of droplets in a respirable range to the respiratory system of a user, the method comprising:
(a) generating an ejected stream of droplets from the fluid composition via a droplet delivery device comprising an ejector mechanism having a piezoelectric actuator, and an aperture plate, the aperture plate having a plurality of openings formed through its thickness and the piezoelectric actuator being operable to directly or indirectly oscillate the aperture plate at a frequency to thereby generate the ejected stream of droplets, wherein at least about 50% of the ejected stream of droplets have an average ejected droplet diameter of less than about 6 μm; and
(b) delivering the ejected stream of droplets to the respiratory system of the user such that at least about 50% of the mass of the ejected stream of droplets is delivered in a respirable range to the respiratory system of a user during use.

21-25. (canceled)
